(19) United States
(12) Patent Application Publication
(10) Pub. No.: US 2015/0239909 A1
(43) Pub. Date: Aug. 27, 2015

(54) PROCESS FOR THE PREPARATION OF (1S,4S,5S)-4-BROMO-6-OXABICYCLO[3.2.1]OCTAN-7-ONE

(71) Applicant: DAIICHI SANKYO COMPANY, LIMITED, Tokyo (JP)

(72) Inventors: Yoshitaka Nakamura, Yokohama (JP); Kumar Madhra Mukesh, Karnal (IN); Murad Ismail Inamdar, Gurgaon (IN)

(73) Assignee: DAIICHI SANKYO COMPANY, LIMITED, Tokyo (JP)

(21) Appl. No.: 14/710,840

(22) Filed: May 13, 2015

Related U.S. Application Data
(63) Continuation of application No. PCT/JP2013/082111, filed on Nov. 22, 2013.

(30) Foreign Application Priority Data
Nov. 23, 2012 (IN) 3605/DEL/2012

(51) Int. Cl. C07D 513/04; C07C 269/00; C07D 307/00

(52) U.S. Cl. CPC C07D 513/04; C07D 307/00; C07C 269/00

(57) ABSTRACT

The present invention relates to an improved and industrially advantageous process for the preparation of (1S, 4S, 5S)-4-bromo-6-oxabicyclo[3.2.1]octan-7-one represented by the following formula (1)

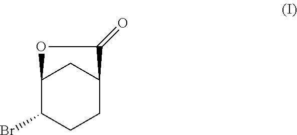

which is a key intermediate in the synthesis of edoxaban, a compound that exhibits on inhibitory effect on activated blood coagulation factor X (also referred to as activated factor X or FXa), and is useful as a preventive and/or therapeutic drug for thrombotic diseases. The process includes reacting (1S)-cyclohex-3-ene-1-carboxylic acid of formula (II) with a brominating agent selected from the group consisting of N-bromosuccinimide or 1,3-dibromo-5, 5-dimethylhydantoin in the presence of a base selected from calcium oxide or calcium hydroxide in a solvent selected from the group comprising of dicholoromethane, toluene, tetrahydrofuran, ethyl acetate, hexanes, cyclopentyl methyl ether (CPME) or a misture thereof to get (1S, 4S 5S)-4-bromo-6-oxabicyclo[3.2.1]octan-y-one of formula (1)

### TECHNICAL FIELD

The present invention relates to an improved and industrially advantageous process for the preparation of (1S,4S,5S)-4-bromo-6-oxabicyclo[3.2.1]octan-7-one of formula (I):

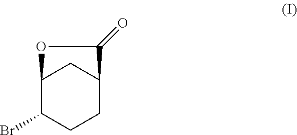

which is a key intermediate in the synthesis of edoxaban, a compound that exhibits an inhibitory effect on activated blood coagulation factor X (also referred to as activated factor X or FXa), and is useful as a preventive and/or therapeutic drug for thrombotic diseases.

### BACKGROUND ART

Chemically, edoxaban is N¹-(5-chloropyridin-2-yl)-N²-((1S,2R,4S)-4-[(dimethylamino)carbonyl]-2-{[(5-methyl-4,5, 6,7-tetrahydrothiazolo[5,4-c]pyridin-2-yl)carbonyl]amino}cyclohexyl) ethanediamide, represented by the following formula (A):

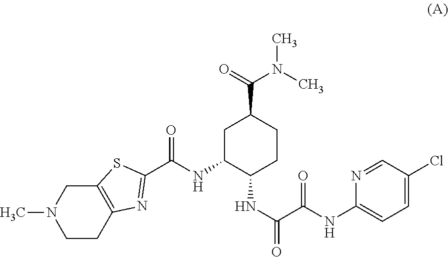 [SMILES: CN1CCc2nc(C(=O)N[C@@H]3C[C@@H](C(=O)N(C)C)CC[C@@H]3NC(=O)C(=O)Nc3ccc(Cl)cn3)sc2C1]

The p-toluenesulfonic acid monohydrate salt of compound A is represented by the following formula (B):

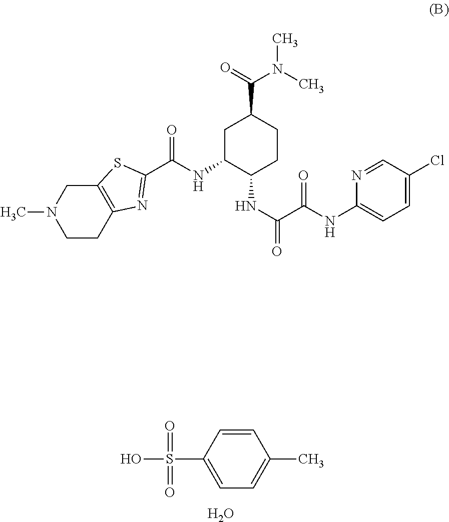

Edoxaban is known as a compound that exhibits an inhibitory effect on activated blood coagulation factor X (also referred to as activated factor X or FXa), and is useful as a preventive and/or therapeutic drug for thrombotic diseases.

Several processes are known in the literature for preparing edoxaban for example, U.S. Pat. No. 7,365,205; U.S. Publication No. 20090105491.

U.S. Pat. No. 7,365,205 provides a process for the preparation of edoxaban, wherein the process involves the use of (1S,4S,5S)-4-iodo-6-oxabicyclo[3.2.1]octan-7-one, represented by the following formula (C):

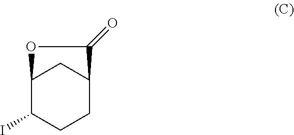

as an intermediate.

The present inventors have identified that (1S,4S,5S)-4-bromo-6-oxabicyclo[3.2.1]octan-7-one, represented by the following formula (I):

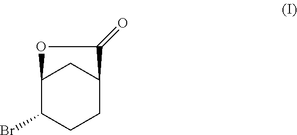

could also be used as an intermediate for the preparation of FXa inhibitory compounds like edoxaban. The present inventors have found that replacement of (1S,4S,5S)-4-iodo-6-oxabicyclo[3.2.1]octan-7-one (C) with (1S,4S,5S)-4-bromo-6-oxabicyclo[3.2.1]octan-7-one (I) has a better atom economy and also an impact on cost.

A method for the synthesis of the (1S,4S,5S)-4-bromo-6-oxabicyclo[3.2.1]octan-7-one (I) was reported in Tetrahedron Letters, 51, (2010) Pages 3433-3435 which involves the reaction of (1S)-cyclohex-3-ene-1-carboxylic acid represented by the following formula (II):

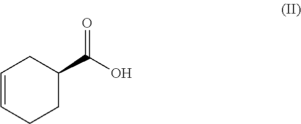

with N-bromosuccinimide in the presence of molecular sieves using dichloromethane as a solvent. However, this reaction is carried out in darkness over a period of 7 hours and does not provide a pure product.

Tetrahedron, Vol. 28, Pages 3393-3399, 1972 provides a process for the preparation of 4-bromo-6-oxabicyclo[3.2.1]octan-7-one which involves the addition of 20% excess of a 2M solution of bromine in chloroform to a stirred solution of cyclohex-3-ene-1-carboxylic acid (0.04 mol) in chloroform (250 mL) in the absence of a base. Extraction with aqueous sodium bicarbonate followed by acidification gave, after extraction with ether and evaporation of the extract, a mixture of cis & trans 3,4-dibromocyclohexanecarboxylic acid (6.7 g) and evaporation of the chloroform layer afforded the bromolactone (0.59 g). It further provides a process for the preparation of 4-bromo-6-oxabicyclo[3.2.1]octan-7-one which involves the treating of cyclohex-3-ene-1-carboxylic acid (0.08 mol) dissolved in chloroform (450 mL) with 20% excess bromine in the presence of an equimolar amount of triethylamine (8.1 g). After extraction of the amine with 2N hydrochloric acid, and work-up, bromolactone (10.7 g) and a mixture of cis & trans 3,4-dibromocyclohexanecarboxylic acid (6.6 g) were obtained.

Tetrahedron Vol. 48, No. 3, Pages 539-544, 1992 provides a process for the preparation of (1S,4S,5S)-4-bromo-6-oxabicyclo[3.2.1]octan-7-one (I) which involves the addition of 1M solution of bromine in chloroform (30 mL) at 0° C. to a solution of (1S)-cyclohex-3-ene-1-carboxylic acid (0.024 mol) of formula (II) in chloroform (600 mL) in the presence of an equimolar amount of triethylamine (3.33 mL). After work-up, the crude bromolactone obtained was recrystallized from petroleum ether.

However, bromination using bromine does not provide a pure product in good yield.

Heterocycles, Vol. 23, No. 8, Pages 2035-2039, 1985 provides a process for the 4-bromo-6-oxabicyclo[3.2.1]octan-7-one which involves the addition of cyclohex-3-ene-1-carboxylic acid (1.0 mM) in 1,2-dimethoxyethane (2 mL) to a stirred solution of 90% Lead (IV) acetate (1.1 or 2.2 mM) in 1,2-dimethoxyethane (4 mL) followed by the addition of Zinc bromide (2.2 mM) in 1,2-dimethoxyethane (4 mL) and continuing the stirring for 10-30 minutes at 0° C. The reaction mixture was poured into a solution of ice-cold water (30 mL) and 10% hydrochloric acid (10 mL), and extracted with ether (50 mL×3). The combined ether extract was washed successively with saturated sodium hydrogen carbonate solution (20 mL), 10% sodium thiosulphate solution (5 mL), and brine (10 mL), and dried over sodium sulphate. Evaporation of the solvent gave crude lactone which was separated and purified (42% yield). However, this reaction does not provide a pure product in good yield.

Heterocycles, Vol. 31, No. 6, Pages 987-991, 1990 provides a method for bromolactonization using a dimethylsulfoxide-trimethylsilyl bromide-amine system. The bromolactonization is carried out for 10 to 72 hours using different solvents and triethylamine or diisopropylethyl amine as base. However, this process does not provide a product in high yield. Further the process afforded the cis isomer exclusively.

Journal of the Chemical Society, Perkin Transactions 1: Organic and Bio-Organic Chemistry (1972-1999) (1994), (7), Pages 847-851 provides a method for bromolactonization using a dimethylsulfoxide-trimethylsilyl bromide-amine system. The bromolactonization is carried out for 12 hours using a dimethylsulfoxide and chloroform solvent system and triethylamine or diisopropylethyl amine as base. However, this process resulted in a low yield of about 55%.

### CITATION LIST

### Patent Literature

PTL1: U.S. Pat. No. 7,365,205
PTL2: U.S. Publication No. 20090105491.

### Non Patent References

NPL1: Feng Chen et al., Tetrahedron Letters, 51, (2010) Pages 3433-3435.
NPL2: G. Belluci et al., Tetrahedron, Vol. 28, No. 13, Pages 3393-3399, 1972.
NPL3: Marco Chini et al., Tetrahedron Vol. 48, No. 3, Pages 539-544, 1992.
NPL4: Y. Fujimoto et al., Heterocycles, Vol. 23, No. 8, Pages 2035-2039, 1985.
NPL5: C. Iwata et al., Heterocycles, Vol. 31, No. 6, Pages 987-991, 1990.
NPL6: K. Miyashita et al., Journal of the Chemical Society, Perkin Transactions 1: Organic and Bio-Organic Chemistry(l972-1999)(1994), (7), Pages 847-851.

### SUMMARY OF INVENTION

### Technical Problem

It is an object of the present invention to solve the problems associated with the prior art, and to provide an improved and efficient method for the preparation of (1S,4S,5S)-4-bromo-6-oxabicyclo[3.2.1]octan-7-one of formula (I).

### Solution to Problem

As a result of conducting diligent studies to attain the object, the present inventors have found that: surprisingly, the use of N-bromosuccinimide or bromohydantoin (representative is 1,3-dibromo-5,5-dimethylhydantoin) as brominating agent in the presence of a base selected from calcium oxide or calcium hydroxide, in specific mole ratios in a solvent selected from the group consisting of dichloromethane, toluene, tetrahydrofuran, ethyl acetate, hexane, cyclopentyl methyl ether (CPME) or a mixture thereof can efficiently produce pure (1S,4S,5S)-4-bromo-6-oxabicyclo[3.2.1]octan-7-one (I) in better yields. The process provides obvious benefits with respect to economics, convenience to operate at a commercial scale.

The present invention also provides replacement of (1S,4S,5S)-4-iodo-6-oxabicyclo[3.2.1]octan-7-one (C) with (1S,4S,5S)-4-bromo-6-oxabicyclo[3.2.1]octan-7-one (I) which has a better atom economy and also an impact on cost in the preparation of edoxaban, a compound that exhibits an inhibitory effect on activated blood coagulation factor X (also referred to as activated factor X or FXa), and is useful as a preventive and/or therapeutic drug for thrombotic diseases.

The present invention provides (1) to (4) shown below.

(1) A process for producing (1S,4S,5S)-4-bromo-6-oxabicyclo[3.2.1]octan-7-one, represented by the following formula (I):

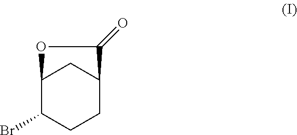

comprising treating (1S)-cyclohex-3-ene-1-carboxylic acid, represented by the following formula (II):

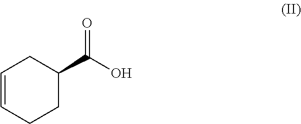

with N-bromosuccinimide or 1,3-dibromo-5,5-dimethylhydantoin as brominating agent in the presence of a base selected from calcium oxide or calcium hydroxide in a solvent selected from the group consisting of dichloromethane, toluene, tetrahydrofuran, ethyl acetate, hexane, cyclopentyl methyl ether (CPME) or a mixture thereof.
(2) The production process according to (1), wherein the brominating agent is N-bromosuccinimide.
(3) A process for producing
tert-butyl{(1R,2S,5S)-2-amino-5-[(dimethylamino)carbonyl]cyclohexyl}carbamate oxalate:
the method using (1S,4S,5S)-4-bromo-6-oxabicyclo[3.2.1]octan-7-one (I) produced by a method according to (1) and comprising the following steps a) and b):

a) treating (1S,4S,5S)-4-bromo-6-oxabicyclo[3.2.1]octan-7-one (I) produced by a production process according to (1) with an aqueous solution of dimethylamine followed by reacting with aqueous ammonia, subsequently with a di-tert-butyl dicarbonate and further with methanesulfonyl chloride to obtain methanesulfonic acid (1R,2R,4S)-2-tert-butoxycarbonylamino-4-dimethylcarbamoyl-cyclohexyl ester,

b) treating the methanesulfonic acid (1R,2R,4S)-2-tert-butoxycarbonylamino-4-dimethylcarbamoyl-cyclohexyl ester with sodium azide, followed by subjecting the resultant compound to hydrogenolysis in the presence of Palladium-Carbon and ammonium formate and reacting the resultant compound with oxalic acid to obtain tert-Butyl{(1R,2S,5S)-2-amino-5-[(dimethylamino)carbonyl]cyclohexyl}carbamate oxalate.

(4) A process for producing N¹-(5-chloropyridin-2-yl)-N²-((1S,2R,4S)-4-[(dimethylamino)carbonyI]-2-{[(5-methyl-4,5, 6,7-tetrahydrothiazolo[5,4-c]pyridin-2-yl)carbonyl]amino}cyclohexyl) ethanediamide p-toluenesulfonate monohydrate:
the method using (1S,4S,5S)-4-bromo-6-oxabicyclo[3.2.1]octan-7-one (I) produced by a method according to (1) and comprising the following steps a) to e):

a) treating (1S,4S,5S)-4-bromo-6-oxabicyclo[3.2.1]octan-7-one (I) produced by a production process according to (1) with an aqueous solution of dimethylamine followed by reacting with aqueous ammonia, subsequently with di-tert-butyl dicarbonate and further with methanesulfonyl chloride to obtain methanesulfonic acid (1R,2R,4S)-2-tert-butoxycarbonylamino-4-dimethylcarbamoyl-cyclohexyl ester,

b) treating the methanesulfonic acid (1R,2R,4S)-2-tert-butoxycarbonylamino-4-dimethylcarbamoyl-cyclohexyl ester with sodium azide, followed by subjecting the resultant compound to hydrogenolysis in the presence of Palladium-Carbon and ammonium formate and reacting the resultant compound with oxalic acid to obtain tert-Butyl{(1R,2S,5S)-2-amino-5-[(dimethylamino)carbonyl]cyclohexyl}carbamate oxalate,

c) reacting the tert-Butyl{(1R,2S,5S)-2-amino-5-[(dimethylamino)carbonyl]cyclohexyl}carbamate oxalate with ethyl[5-chloropyridin-2-yl]amino](oxo)acetate hydrochloride in the presence of triethylamine to obtain tert-Butyl[(1R,2S,5S)-2-({[(5-chloropyridin-2-yl)amino](oxo)acetyl}amino)-5-(dimethyla minocarbonyl)cyclohexyl]carbamate,

d) deprotecting the tert-Butoxycarbonyl group from the tert-Butyl[(1R,2S,5S)-2-({[(5-chloropyridin-2-yl)amino](oxo)acetyl}amino)-5-(dimethyla minocarbonyl)cyclohexyl]carbamate by using methanesulphonic acid followed by reacting the deprotected compound with 5-methyl-4,5,6,7-tetrahydro[1,3]thiazolo[5,4-c]pyridine-2-carboxylic acid hydrochloride or its activated ester to obtain N¹-(5-chloropyridin-2-yl)-N²-((1S,2R,4S)-4-[(dimethylamino)carbonyl]-2-{[(5-methyl-4,5, 6,7-tetrahydrothiazolo[5,4-c]pyridin-2-yl)carbonyl]amino}cyclohexyl) ethanediamide,

e) treating the N¹-(5-chloropyridin-2-yl)-N²-((1S,2R,4S)-4-[(dimethylamino)carbonyl]-2-{[(5-methyl-4,5, 6,7-tetrahydrothiazolo[5,4-c]pyridin-2-yl)carbonyl]amino}cyclohexyl) ethanediamide with p-toluenesulfonic acid in aqueous ethanol to obtain N¹-(5-chloropyridin-2-yl)-N²-((1S,2R,4S)-4-[(dimethylamino)carbonyl]-2-{[(5-methyl-4,5, 6,7-tetrahydrothiazolo[5,4-c]pyridin-2-yl)carbonyl]amino}cyclohexyl) ethanediamide p-toluenesulfonate monohydrate.

### Advantageous Effects of Invention

The present invention provides a novel method for an improved and efficient method for the preparation of (1S,4S,5S)-4-bromo-6-oxabicyclo[3.2.1]octan-7-one (I) which has a better atom economy and also an impact on cost in the preparation of edoxaban, a compound that exhibits an inhibitory effect on activated blood coagulation factor X (also referred to as activated factor X or FXa), and is useful as a preventive and/or therapeutic drug for thrombotic diseases.

### DESCRIPTION OF EMBODIMENTS

More particularly, the present invention relates to a process for the preparation of (1S,4S,5S)-4-bromo-6-oxabicyclo[3.2.1]octan-7-one, represented by the following formula (I):

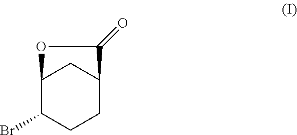

which comprises reacting (1S)-cyclohex-3-ene-1-carboxylic acid, represented by the following formula (II):

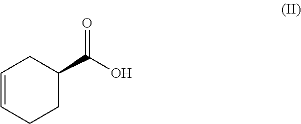

with a brominating agent selected from the group consisting of N-bromosuccinimide or 1,3-dibromo-5,5-dimethylhydantoin in the presence of a base selected from calcium oxide or calcium hydroxide in a solvent selected from the group comprising of dichloromethane, toluene, tetrahydrofuran, ethyl acetate, hexane, cyclopentyl methyl ether (CPME) or a mixture thereof.

The process further includes the optional step of recrystallization of (1S,4S,5S)-4-bromo-6-oxabicyclo[3.2.1]octan-7-one (I) from a single or a mixed solvent system.

The starting material, (1S)-cyclohex-3-ene-1-carboxylic acid of formula (II) may be prepared according to the methods provided in the art for example as per U.S. 2011/0257401.

N-Bromosuccinimide is suitably used in an amount of 1.0 to 1.1 molar equivalents of compound of formula (II), preferably in an amount of 1.02 to 1.08 molar equivalents and more preferably in an amount of 1.05 molar equivalents of compound of formula (II). The present inventors have found that the quality of N-Bromosuccinimide also has an impact on the purity of (1S,4S,5S)-4-bromo-6-oxabicyclo[3.2.1]octan-7-one (I). The present inventors have found that (1S,4S,5S)-4-bromo-6-oxabicyclo[3.2.1]octan-7-one (I) prepared by using unpurified N-bromosuccinimide contains an impurity of about 5.6% which corresponds to the methoxy derivative of N-bromosuccinimide.

1,3-Dibromo-5,5-dimethylhydantoin is suitably used in an amount of 0.5 to 0.6 molar equivalents of compound of formula (II), preferably in an amount of 0.51 to 0.55 molar equivalents and more preferably in an amount of 0.52 molar equivalents of compound of formula (II).

Calcium oxide is suitably used in an amount of 0.07 to 0.13 molar equivalents of compound of formula (II), preferably in an amount of 0.08 to 0.12 molar equivalents and more preferably in an amount of 0.1 molar equivalents of compound of formula (II).

Calcium hydroxide is suitably used in an amount of 0.05 to 0.5 molar equivalents of compound of formula (II).

The reaction of (1S)-cyclohex-3-ene-1-carboxylic acid of formula (II) with a brominating agent selected from the group consisting of N-bromosuccinimide or 1,3-dibromo-5,5-dimethylhydantoin in the presence of a base selected from calcium oxide or calcium hydroxide is carried out at a selected temperature range of 15 to 40° C., preferably at 20 to 25° C., during a period of 15 minutes to several hours, preferably for about 15 minutes to 1 hour.

In an embodiment, a solution of (1S)-cyclohex-3-ene-1-carboxylic acid of formula (II) in a solvent selected from the group comprising of dichloromethane, toluene, tetrahydrofuran, ethyl acetate, hexane, cyclopentyl methyl ether (CPME) or a mixture thereof is added in a drop-wise manner or in lots to the reaction mixture comprising a brominating agent selected from the group consisting of N-bromosuccinimide or 1,3-dibromo-5,5-dimethylhydantoin, a base selected from calcium oxide or calcium hydroxide in a solvent selected from the group comprising of dichloromethane, toluene, tetrahydrofuran, ethyl acetate, hexane, cyclopentyl methyl ether (CPME) or a mixture thereof.

(1S,4S,5S)-4-Bromo-6-oxabicyclo[3.2.1]octan-7-one of formula (I) may be isolated by a common isolation technique such as cooling, extraction, one or more of washing, crystallization, precipitation, filtration, filtration under vacuum, decantation and centrifugation or a combination thereof.

(1S,4S,5S)-4-Bromo-6-oxabicyclo[3.2.1]octan-7-one of formula (I) may be isolated from the reaction mixture by optionally adding water to the reaction mixture followed by filtration and/or concentration followed by isolation from water.

More preferably, the desired compound, (1S,4S,5S)-4-bromo-6-oxabicyclo[3.2.1]octan-7-one of formula (I) is isolated by complete removal of the solvent from the organic layer and the solid thus separated is charged with pre-heated water at 50° C., stirred for 15 minutes at 50±2° C., filtered and dried to get the pure desired compound.

The compound of formula (I) is optionally dried further and/or recrystallized from a single or a mixed solvent system. The solvent may be an organic solvent selected from the group consisting of alcohols, ketones, ethers or a mixture thereof.

In an embodiment, (1S,4S,5S)-4-bromo-6-oxabicyclo[3.2.1]octan-7-one of formula (I) may be recrystallized from acetone and water. The recrystallization of the compound of formula (I) comprises the steps of a) providing a solution of (1S,4S,5S)-4-bromo-6-oxabicyclo[3.2.1]octan-7-one (I) in acetone, b) combining the solution obtained in step a) with water, and c) isolating (1S,4S,5S)-4-bromo-6-oxabicyclo[3.2.1]octan-7-one (I).

Step a) of providing a solution of (1S,4S,5S)-4-bromo-6-oxabicyclo[3.2.1]octan-7-one (I) in acetone includes dissolving (1S,4S,5S)-4-bromo-6-oxabicyclo[3.2.1]octan-7-one (I) in acetone at a temperature of about 45° C. to 60° C. optionally under stirring.

Step b) involves combining the solution obtained in step a) with water. The term “combining” includes adding, dissolving, slurrying, stirring, or a combination thereof. The water can be added at about 40 to 60° C., preferably at 40 to 50° C. during a period of 15 minutes to several hours, preferably for about 15 minutes to 2 hours, followed by stirring the reaction mass at 0 to 8° C., preferably at 5 to 8° C. for a period of 15 minutes to 2 hours, preferably for about 15 minutes to 1 hour.

In step c) (1S,4S,5S)-4-bromo-6-oxabicyclo[3.2.1]octan-7-one (I) can be isolated by a common isolation technique such as cooling, extraction, one or more of washing, crystallization, precipitation, filtration, filtration under vacuum, decantation and centrifugation or a combination thereof.

(1S,4S,5S)-4-Bromo-6-oxabicyclo[3.2.1]octan-7-one (I) thus synthesized is useful as an intermediate for the preparation of edoxaban or its pharmaceutically acceptable salts or hydrates thereof, a compound that exhibits an inhibitory effect on activated blood coagulation factor X (also referred to as activated factor X or FXa), and is useful as a preventive and/or therapeutic drug for thrombotic diseases.

Edoxaban of formula (A) or p-toluenesulfonic acid monohydrate salt of compound A of formula (B) as disclosed in, for example, U.S. Pat. No. 7,365,205 and U.S. Publication No. 20090105491, may be produced from (1S,4S,5S)-4-bromo-6-oxabicyclo[3.2.1]octan-7-one of formula (I) prepared as per the present invention, in accordance with process steps as described herein, or as described in, for example, U.S. Publication No. 20090105491 and U.S. Pat. No. 7,365,205.

The steps comprise of:

a) treating (1S,4S,5S)-4-bromo-6-oxabicyclo[3.2.1]octan-7-one of (I) with an aqueous solution of dimethylamine followed by reacting with aqueous ammonia, subsequently with a di-tert-butyl dicarbonate and further with methanesulfonyl chloride to obtain methanesulfonic acid (1R,2R,4S)-2-tert-butoxycarbonylamino-4-dimethylcarbamoyl-cyclohexyl ester,

b) treating the methanesulfonic acid (1R,2R,4S)-2-tert-butoxycarbonylamino-4-dimethylcarbamoyl-cyclohexyl ester with sodium azide, followed by subjecting the resultant compound to hydrogenolysis in the presence of Palladium-Carbon and ammonium formate and reacting the resultant compound with oxalic acid to obtain tert-Butyl{(1R,2S,5S)-2-amino-5-[(dimethylamino)carbonyl]cyclohexyl}carbamate oxalate,

c) reacting the tert-Butyl{(1R,2S,5S)-2-amino-5-[(dimethylamino)carbonyl]cyclohexyl}carbamate oxalate with ethyl[5-chloropyridin-2-yl]amino](oxo)acetate hydrochloride in the presence of a triethylamine to obtain tert-Butyl[(1R,2S,5S)-2-({[(5-chloropyridin-2-yl)amino](oxo)acetyl}amino)-5-(dimethyla minocarbonyl)cyclohexyl]carbamate,

d) deprotecting the tert-Butoxycarbonyl group from the tert-Butyl[(1R,2S,5S)-2-({[(5-chloropyridin-2-yl)amino](oxo)acetyl}amino)-5-(dimethyla minocarbonyl)cyclohexyl]carbamate by using methanesulphonic acid followed by reacting the deprotected compound with 5-methyl-4,5,6,7-tetrahydro[1,3]thiazolo[5,4-c]pyridine-2-carboxylic acid hydrochloride or its activated ester to obtain edoxaban, and optionally,

e) converting the edoxaban to its pharmaceutically acceptable salts or hydrates thereof, for example, treating the edoxaban with p-toluenesulfonic acid in aqueous ethanol as solvent to obtain edoxaban p-toluenesulfonate monohydrate (salt and hydrate form) of formula (B).

While the present invention has been described in terms of its specific embodiments, certain modifications and equivalents will be apparent to those skilled in the art and are intended to be included within the scope of the present invention.

### EXAMPLES

### Example 1

### Preparation of (1S,4S,5S)-4-Bromo-6-oxabicyclo[3.2.1]octan-7-one (I)

Controlled addition of (1S)-Cyclohex-3-ene-1-carboxylic acid to a mixture of N-bromosuccinimide and calcium oxide

Solution A: To a suspension of dichloromethane (150 mL), water (90 mL), and (1S)-cyclohex-3-ene-1-carboxylic acid-(R)-phenyl ethyl amine salt (30 g), conc. hydrochloric acid (35%, 13.9 mL) was added. The reaction mass was stirred for 15 minutes and the layers were separated. The aqueous layer was extracted with dichloromethane (90 mL). The combined organic layer was washed with water (90 mL) and recovered under vacuum at 35° C. to afford an oil. Dichloromethane (75 mL) was charged to the above oil to get Solution A.

Solution B: N-Bromosuccinimide (22.22 g) and calcium oxide (0.6 g) were dissolved in dichloromethane (30 mL) to get Solution B.

Solution A of (1S)-cyclohex-3-ene-1-carboxylic acid (II) in dichloromethane (75 mL) was added drop-wise to Solution B in a time period of 1 hour at 23±3° C. The reaction mass was stirred for 1 hour at 23±3° C., filtered through a Hyflo bed and washed with dichloromethane (30 mL). The filtrate was recovered under vacuum at 34° C. to get a solid. Pre-heated water (75 mL) was added to the above solid and the reaction mass was stirred at 50° C. for 15 minutes. The solid was filtered, washed with pre-heated water (30 mL) and isolated. Pre-heated water (75 mL) was again charged to the above solid and the reaction mass was stirred at 50° C. for 15 minutes. The solid was filtered and washed with pre-heated water (30 mL). The solid was dried under vacuum at 35 to 40° C. for 14 hours to get the title compound (I).

### Yield: 84.43%

Chromatographic purity: 99.56%

### Example 2

### Preparation of (1S,4S,5S)-4-Bromo-6-oxabicyclo[3.2.1]octan-7-one (I)

Addition of N-Bromosuccinimide and calcium oxide to (1S)-Cyclohex-3-ene-1-carboxylic acid in lots

(1S)-Cyclohex-3-ene-1-carboxylic acid (II) (5 g) was dissolved in dichloromethane (25 mL). To this solution N-bromosuccinimide (1.1 mole) was added at room temperature. Calcium oxide (0.25 mole) was charged to the suspension in two lots. The reaction mixture was stirred at 20 to 25° C. for 1 hour and filtered. The bed was washed with dichloromethane (10 mL). The washings were combined with the filtrate and the solvent was recovered under vacuum at 35 to 40° C. Deionized water (50 mL) was charged to the solid, heated to 50° C., stirred for 10 minutes and filtered. The bed was washed with deionized water (10 mL) and suction dried. The solid was dried under vacuum at 45-50° C. to get the title compound (I).

### Yield: 61%

Chromatographic purity: 96.98%

### Example 3

### Preparation of (1S,4S,5S)-4-Bromo-6-oxabicyclo[3.2.1]octan-7-one (I)

(1S)-Cyclohex-3-ene-1-carboxylic acid (II) (20.4 g) was dissolved in dichloromethane (100 mL). This solution was added to a solution of N-bromosuccinimide (30.22 g) and calcium oxide (0.906 g) dissolved in dichloromethane (40 mL) in 30 minutes at room temperature. The reaction mass was stirred for 30 minutes and filtered. The filtrate was concentrated to give a solid. Deionized water (100 mL) was added to the solid and heated to 50° C. and stirred for 15 minutes. The solid was filtered and recharged into a reaction flask. Deionized water (100 mL) was added to the solid, heated to 50° C. and stirred for 15 minutes. The solid was filtered and dried under vacuum to obtain the title compound (I).

### Yield: 77.7

Chromatographic purity: 96.11
Water content: 0.02% w/w

### Example 4

### Preparation of (1S,4S,5S)-4-Bromo-6-oxabicyclo[3.2.1]octan-7-one (I)

1S)-Cyclohex-3-ene-1-carboxylic acid (II) (20.4 g) was dissolved in ethyl acetate (100 mL). This solution was added to a solution of N-bromosuccinimide (30.22 g) and calcium oxide (0.906 g) dissolved in ethyl acetate (40 mL) in 30 minutes at room temperature. The reaction mass was stirred for 2 hours, filtered to obtain a residue. The filtrate was concentrated to give a solid. Deionized water (100 mL) was added to the solid, heated to 50° C. and stirred for 15 minutes. The solid was filtered and recharged into a reaction flask. Deionized water (100 mL) was added to the solid, heated to at 50° C. and stirred for 15 minutes. The solid was filtered and dried under vacuum to obtain the title compound.

Dichloromethane (100 mL) was added to the residue, stirred for 10 minutes and filtered. The filtrate was concentrated to give a solid. Deionized water (50 mL) was added to the solid, heated to 50±2° C. and stirred for 15 minutes. The solid was filtered and recharged into a reaction flask. Deionized water (50 mL) was added to the solid, heated to 50° C. and stirred for 15 minutes. The solid was filtered and dried under vacuum to obtain the title compound (I).

Combined yield: 67.67%
Chromatographic purity of combined solids: 95.74
Water content of combined solids: 0.03% w/w

### Example 5

### Preparation of (1S,4S,5S)-4-Bromo-6-oxabicyclo[3.2.1]octan-7-one (I)

(1S)-Cyclohex-3-ene-1-carboxylic acid (II) (20.4 g) was dissolved in toluene (100 mL). This solution was added to a solution of N-bromosuccinimide (30.22 g) and calcium oxide (0.906 g) dissolved in toluene (40 mL) in 25 minutes at 18 to 33C. The reaction mass was stirred for 1 hour and filtered to obtain a residue. The filtrate was concentrated to give a solid. Dichloromethane (100 mL) was added to the residue, stirred for 10 minutes, filtered and the filtrate was concentrated to give a solid. The solids were combined and deionized water (100 mL) was added to the solid, heated to 50° C. and stirred for 15 minutes. The solid was filtered and recharged into a reaction flask. Deionized water (100 mL) was added to the solid, heated to 50° C. and stirred for 15 minutes. The solid was filtered and dried under vacuum to obtain the title compound (I).

### Yield: 79.31%

Chromatographic purity: 81.54%
Water content: 0.06% w/w

### Example 6

### Preparation of (1S,4S,5S)-4-Bromo-6-oxabicyclo[3.2.1]octan-7-one (I)

(1S)-Cyclohex-3-ene-1-carboxylic acid (II) (20.4 g) was dissolved in tetrahydrofuran (100 mL). This solution was added to a solution of N-bromosuccinimide (30.22 g) and calcium oxide (0.906 g) dissolved in tetrahydrofuran (40 mL) in 35 minutes at 20° C. The reaction mass was stirred for 1 hour, filtered and the filtrate was concentrated to give a solid. Deionized water (100 mL) was added to the solid, heated to 50° C. and stirred for 15 minutes. The solid was filtered and recharged into a reaction flask. Deionized water (100 mL) was added to the solid and heated to 50° C. and stirred for 15 minutes. The solid was filtered and dried under vacuum to obtain the title compound (I).

### Yield: 66.34%

Chromatographic purity: 89.95%
Water content: 0.04% w/w

### Example 7

### Preparation of (1S,4S,5S)-4-Bromo-6-oxabicyclo[3.2.1]octan-7-one (I)

(1S)-Cyclohex-3-ene-1-carboxylic acid (II) (20.4 g) was dissolved in cyclopentyl methyl ether (100 mL). This solution was added to a solution of N-bromosuccinimide (30.22 g) and calcium oxide (0.906 g) dissolved in cyclopentyl methyl ether (CPME)(40 mL) in 35 minutes at 20 to 33° C. The reaction mass was stirred for 1 hour, filtered to obtain a residue. Dichloromethane (100 mL) was added to the residue, stirred for 10 minutes and filtered. The filtrates were combined and the solvent was recovered under vacuum. Deionized water (100 mL) was added to the solid, heated to 50° C. and stirred for 15 minutes. The solid was filtered and recharged into a reaction flask. Deionized water (100 mL) was added to the solid, heated to 50° C. and stirred for 15 minutes. The solid was filtered and dried under vacuum to obtain the title compound (I)

### Yield: 74.63%

Chromatographic purity: 94.17%
Water content: 0.02% w/w

### Example 8

### Preparation of (1S,4S,5S)-4-Bromo-6-oxabicyclo[3.2.1]octan-7-one (I)

(1S)-Cyclohex-3-ene-1-carboxylic acid (II) (10.2 g) was dissolved in dichloromethane (50 mL). This solution was added to a solution of N-bromosuccinimide (0.979 mole) and calcium hydroxide (0.49 mole) dissolved in dichloromethane (20 mL) in 50 minutes. The reaction mass was stirred for 1 hour at 30 to 35° C. and filtered through a Hyflo bed. The bed was washed with dichloromethane (20 mL). The filtrate was concentrated under vacuum at 35° C. to give a solid. The solid obtained was washed twice by making slurry in water (50 mL) at 50° C. The solid was filtered and dried under vacuum at 45° C. for 14 hours to obtain the title compound (I).

### Yield: 70%

Chromatographic purity: 96.13%

### Example 9

### Preparation of (1S,4S,5S)-4-Bromo-6-oxabicyclo[3.2.1]octan-7-one (I)

(1S)-Cyclohex-3-ene-1-carboxylic acid (II) (10.4 g) was dissolved in toluene (50 mL). This solution was added to a solution of 1,3-dibromo-5,5-dimethylhydantoin (0.55 mole) and calcium oxide (0.1 mole) dissolved in toluene (20 mL) at 20 to 25° C. in 60 minutes. The reaction mass was stirred for 1 hour at 20 to 25° C. and filtered. The bed was washed with toluene (50 mL) and dichloromethane (50 mL). The filtrate was concentrated under vacuum to give a solid. Deionized water (50 mL) was added to the solid, heated to 50° C. and stirred for 15 minutes. The solid was filtered and recharged into a reaction flask. Deionized water (50 mL) was added to the solid, heated to 50° C. and stirred for 15 minutes. The solid was filtered and dried under vacuum to obtain the title compound (I).

### Yield: 78.31%

Chromatographic purity: 66.89%

### Example 10

### Preparation of (1S,4S,5S)-4-Bromo-6-oxabicyclo[3.2.1]octan-7-one (I)

(1S)-Cyclohex-3-ene-1-carboxylic acid (II) (20.6 g) was dissolved in toluene (100 mL). This solution was added to a solution of 1,3-dibromo-5,5-dimethylhydantoin (0.52 mole) and calcium hydroxide (0.09 mole) dissolved in toluene (40 mL) at 20 to 30° C. in 45 minutes. The reaction mass was stirred for 1 hour at 25 to 30° C. and filtered. The bed was washed with toluene (40 mL) and the filtrate was concentrated under vacuum to give a solid. The bed was again washed with toluene (200 mL) and the filtrate was concentrated under vacuum to give a solid. The solids were combined. Deionized water (100 mL) was added to the solid, heated to 50° C. and stirred for 15 minutes. The solid was filtered and recharged into a reaction flask. Deionized water (100 mL) was added to the solid, heated to 50° C. and stirred for 15 minutes. The solid was filtered and dried under vacuum to obtain the title compound (I).

### Yield: 78.4%

Chromatographic purity: 75.21%

### Example 11

### Purification of (1S,4S,5S)-4-Bromo-6-oxabicyclo[3.2.1]octan-7-one (I)

(1S,4S,5S)-4-bromo-6-oxabicyclo[3.2.1]octan-7-one (I) (120 g) was added to acetone (480 mL) at 45° C. and stirred at 45 to 47° C. to get a clear solution. Water (720 mL) was charged drop-wise at 45 to 50° C. in a time period of 40 minutes. The reaction mass was cooled to 5° C. under stirring and further stirred for 1 hour at 5 to 8° C. The solid was filtered and washed with a mixture of acetone (48 mL) and water (72 mL). The product was suction dried on the Buchner funnel and then in vacuum dryer under vacuum at 40 to 45° C. to obtain the title compound (I).

Yield: 112 g (dry weight) (93.3%)
Chromatographic Purity of crude: 82.46%
Chromatographic Purity of pure: 98.96%

### Example 12

### Preparation of Methanesulfonic acid (1R,2R,4S)-2-tert-butoxycarbonylamino-4-dimethylcarbamoyl-cyclohexyl ester

(1S,4S,5S)-4-Bromo-6-oxabicyclo[3.2.1]octan-7-one (20 g) was dissolved in acetonitrile (125 ml) and 50% dimethylamine aqueous solution (35.2 g) was added to the mixture at around 10° C. The reaction mass was stirred for 15 hours at around 10° C. and the solvent was recovered under vacuum at a temperature less than 15° C. 28% Ammonia solution (125 ml) was added to the residue. The resulting mixture was warmed to 35 to 45° C. for 8 hours and further stirred at about 25° C. for 14 hours. The solvent was recovered under vacuum. Deionized water (63 ml) was added to the residue, and the mixture was concentrated again under vacuum. Deionized water (88 ml), di-tert-butyl dicarbonate (31.9 g) and 48% sodium hydroxide (20.3 g) were added to the residue, and the resulting mixture was stirred at 40 to 45° C. for 2 hours. 4-Methyl-2-pentanone (175 ml) was added to the reaction mass and the layers were separated. The aqueous layer was extracted with 4-methyl-2-pentanone (175 ml). The organic layers were combined, and the solvent was recovered under vacuum until the total volume was about 175 ml. 4-Methyl-2-pentanone (100 ml) was added to the residue, and the mixture was concentrated again to about 175 ml under vacuum. Then, the volume of the solution was adjusted to 250 ml by adding 4-Methyl-2-pentanone. After adding methanesulfonyl chloride (17.9 g) to the solution, triethylamine (18.8 g) was slowly added to the mixture at 15-30° C., and the reaction mass was stirred for 1 hour at the same temperature. After the completion of the reaction, methanol (43 ml) and Deionized water (63 ml) were added to the reaction mass, and the resulting mixture was stirred for 15 minutes. The organic layer was separated, washed with 5% aqueous sodium bicarbonate solution (50 ml), and concentrated under vacuum to adjust the volume to 100 ml. Then, the resulting slurry was stirred for 3 hours at around 0° C. The precipitates were collected by filtration, washed with 4-methyl-2-pentanone (25 ml), and dried under vacuum to give the title compound.

### Yield: 22.4 g (62.9%)

Chromatographic purity: 99.23%

### REFERENTIAL EXAMPLES

### Referential Example 1

### tert-Butyl{(1R,2S,5S)-2-amino-5-[(dimethylamino)carbonyl]cyclohexyl}carbamate oxalate

Sodium azide (7.14 g) and dodecylpyridinium chloride (7.80 g) were added at room temperature to a solution (100 mL) of Methanesulfonic acid (1R,2R,4S)-2-tert-butoxycarbonylamino-4-dimethylcarbamoyl-cyclohexyl ester (20.0 g) in toluene, followed by stirring at 60° C. for 72 hours. Water was added to the reaction mixture, and the organic layer was washed with aqueous saturated sodium bicarbonate solution and water. Methanol, 7.5% Pd-C, and ammonium formate were added to the washed organic layer, followed by stirring at 40° C. for 1 hour. Pd-C was removed by filtration, and the solvent was evaporated under reduced pressure. Hydrated acetonitrile (200 mL) and anhydrous oxalic acid (4.94 g) were added to the residue. The mixture was stirred at room temperature for 17 hours, and the formed crystals were collected by filtration. Acetonitrile (200 mL) was added to the collected crystals, followed by stirring at 40° C. for 24 hours. The formed crystals were recovered by filtration and dried, to thereby yield 12.7 g of the title compound.

### Referential Example 2

### tert-Butyl[(1R,2S,5S)-2-({[(5-chloropyridin-2-yl)amino](oxo)acetyl}amino)-5-(dimethylaminocarbonyl)cyclohexyl]carbamate

Triethylamine (169 mL) was added at 60° C. to a suspension (550 mL) of tert-butyl {(1R,2S,5S)-2-amino-5-[(dimethylamino)carbonyl]cyclohexyl}carbamate oxalate (100.1 g) in acetonitrile. At the same temperature, ethyl [5-chloropyridin-2-yl]amino](oxo)acetate hydrochloride (84.2 g) was added to the mixture, followed by stirring for 6 hours. Thereafter, the mixture was stirred at room temperature for 16 hours. Water was added to the reaction mixture, followed by stirring at 10° C. for 1.5 hours. The formed crystals were recovered by filtration, to thereby yield 106.6 g of the title compound.

### Referential Example 3

### N¹-(5-Chloropyridin-2-yl)-N²-[(1S,2R,4S)-4-(dimethylcarbamoyl)-2-{[(5-methyl-4,5,6,7-tetrahydro[1,3]thiazolo[5,4-c]pyridin-2-yl)carbonyl]amino}cyclohexyl]ethanediamide (Edoxaban)

Methanesulfonic acid (66 mL) was added at room temperature to a suspension of tert-butyl[(1R,2S,5S)-2-({[(5-chloropyridin-2-yl)amino](oxo)acetyl}amino)-5-(dimethylaminocarbonyl)cyclohexyl]carbamate (95.1 g) in acetonitrile (1,900 mL), and the mixture was stirred at the same temperature for 2 hours. Triethylamine (155 mL), 5-methyl-4,5,6,7-tetrahydro[1,3]thiazolo[5,4-c]pyridine-2-carboxylic acid hydrochloride (52.5 g), 1-hydroxybenzotriazole (33.0 g), and 1-ethyl-3-(3-dimethylaminopropyl)carbodiimide hydrochloride (46.8 g) were added to the reaction mixture under ice-cooling, and the mixture was stirred at room temperature for 16 hours. Triethylamine and water were added thereto, followed by stirring under ice-cooling for 1 hour. The formed crystals were recovered by filtration, to thereby yield 103.2 g of the title compound.

### INDUSTRIAL APPLICABILITY

The present invention can be used as an industrial process for producing an (1S,4S,5S)-4-bromo-6-oxabicyclo[3.2.1]octan-7-one of formula (I) which is a key intermediate in the synthesis of edoxaban, a compound that exhibits an inhibitory effect on activated blood coagulation factor X (also referred to as activated factor X or FXa), and is useful as a preventive and/or therapeutic drug for thrombotic diseases.

1. A process for producing (1S,4S,5S)-4-bromo-6-oxabicyclo[3.2.1]octan-7-one represented by the following formula (I): 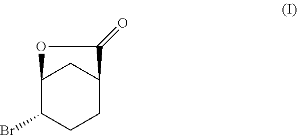
comprising treating (1S)-cyclohex-3-ene-1-carboxylic acid represented by the following formula (II): 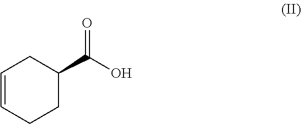
with N-bromosuccinimide or 1,3-dibromo-5,5-dimethylhydantoin as brominating agent in the presence of a base selected from calcium oxide or calcium hydroxide in an organic solvent.
(1) A process for producing (1S,4S,5S)-4-bromo-6-oxabicyclo[3.2.1]octan-7-one, represented by the following formula (I): 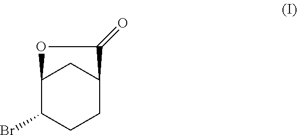
comprising treating (1S)-cyclohex-3-ene-1-carboxylic acid, represented by the following formula (II): 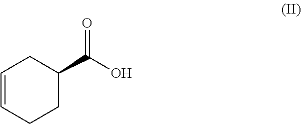
with N-bromosuccinimide or 1,3-dibromo-5,5-dimethylhydantoin as brominating agent in the presence of a base selected from calcium oxide or calcium hydroxide in a solvent selected from the group consisting of dichloromethane, toluene, tetrahydrofuran, ethyl acetate, hexane, cyclopentyl methyl ether (CPME) or a mixture thereof.

2. The production process according to claim 1, wherein the brominating agent is N-bromosuccinimide.

3. A process for producing tert-butyl{(1R,2S,5S)-2-amino-5-[(dimethylamino)carbonyl]cyclohexyl}carbamate oxalate:
the method using (1S,4S,5S)-4-bromo-6-oxabicyclo[3.2.1]octan-7-one (I) produced by a method according to claim 1 and comprising the following steps a) and b):
a) treating (1S,4S,5S)-4-bromo-6-oxabicyclo[3.2.1]octan-7-one (I) produced by a production process according to (1) with an aqueous solution of dimethylamine followed by reacting with aqueous ammonia, subsequently with a di-tert-butyl dicarbonate and further with methanesulfonyl chloride to obtain methanesulfonic acid (1R,2R,4S)-2-tert-butoxycarbonylamino-4-dimethylcarbamoyl-cyclohexyl ester,
b) treating the methanesulfonic acid (1R,2R,4S)-2-tert-butoxycarbonylamino-4-dimethylcarbamoyl-cyclohexyl ester with sodium azide, followed by subjecting the resultant compound to hydrogenolysis in the presence of Palladium-Carbon and ammonium formate and reacting the resultant compound with oxalic acid to obtain tert-Butyl{(1R,2S,5S)-2-amino-5-[(dimethylamino)carbonyl]cyclohexyl}carbamate oxalate.

4. A process for producing N¹-(5-chloropyridin-2-yl)-N²-((1S,2R,4S)-4-[(dimethylamino)carbonyl]-2-{[(5-methyl-4,5, 6,7-tetrahydrothiazolo[5,4-c]pyridin-2-yl)carbonyl]amino}cyclohexyl) ethanediamide p-toluenesulfonate monohydrate:
the method using (1S,4S,5S)-4-bromo-6-oxabicyclo[3.2.1]octan-7-one (I) produced by a method according to claim 1 and comprising the following steps a) to e):
a) treating (1S,4S,5S)-4-bromo-6-oxabicyclo[3.2.1]octan-7-one (I) produced by a production process according to (1) with an aqueous solution of dimethylamine followed by reacting with aqueous ammonia, subsequently with di-tert-butyl dicarbonate and further with methanesulfonyl chloride to obtain methanesulfonic acid (1R,2R,4S)-2-tert-butoxycarbonylamino-4-dimethylcarbamoyl-cyclohexyl ester,
b) treating the methanesulfonic acid (1R,2R,4S)-2-tert-butoxycarbonylamino-4-dimethylcarbamoyl-cyclohexyl ester with sodium azide, followed by subjecting the resultant compound to hydrogenolysis in the presence of Palladium-Carbon and ammonium formate and reacting the resultant compound with oxalic acid to obtain tert-Butyl{(1R,2S,5S)-2-amino-5-[(dimethylamino)carbonyl]cyclohexyl}carbamate oxalate,
c) reacting the tert-Butyl{(1R,2S,5S)-2-amino-5-[(dimethylamino)carbonyl]cyclohexyl}carbamate oxalate with ethyl[5-chloropyridin-2-yl]amino](oxo)acetate hydrochloride in the presence of triethylamine to obtain tert-Butyl[(1R,2S,5S)-2-({[(5-chloropyridin-2-yl)amino](oxo)acetyl}amino)-5-(dimethylaminocarbonyl)cyclohexyl]carbamate,
d) deprotecting the tert-Butoxycarbonyl group from the tert-Butyl[(1R,2S,5S)-2-({[(5-chloropyridin-2-yl)amino](oxo)acetyl}amino)-5-(dimethylaminocarbonyl)cyclohexyl]carbamate by using methanesulphonic acid followed by reacting the deprotected compound with 5-methyl-4,5,6,7-tetrahydro[1,3]thiazolo[5,4-c]pyridine-2-carboxylic acid hydrochloride or its activated ester to obtain N¹-(5-chloropyridin-2-yl)-N²-((1S,2R,4S)-4-[(dimethylamino)carbonyl]-2-{[(5-methyl-4,5, 6,7-tetrahydrothiazolo[5,4-c]pyridin-2-yl)carbonyl]amino}cyclohexyl) ethanediamide,
e) treating the N¹-(5-chloropyridin-2-yl)-N²-((1S,2R,4S)-4-[(dimethylamino)carbonyl]-2-{[(5-methyl-4,5, 6,7-tetrahydrothiazolo[5,4-c]pyridin-2-yl)carbonyl]amino}cyclohexyl) ethanediamide with p-toluenesulfonic acid in aqueous ethanol to obtain N¹-(5-chloropyridin-2-yl)-N²-((1S,2R,4S)-4-[(dimethylamino)carbonyl]-2-{[(5-methyl-4,5, 6,7-tetrahydrothiazolo[5,4-c]pyridin-2-yl)carbonyl]amino}cyclohexyl) ethanediamide p-toluenesulfonate monohydrate.
